(19) United States
(12) Patent Application Publication
(10) Pub. No.: US 2005/0282816 A1
(43) Pub. Date: Dec. 22, 2005

(54) Pyrazinylmethyl lactam derivatives

(75) Inventor: Christopher J. Helal, Mystic, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/135,186

(22) Filed: May 23, 2005

Related U.S. Application Data
(60) Provisional application No. 60/573,559, filed on May 21, 2004.

(51) Int. Cl. A61K 31/497; C07D 43/02

(52) U.S. Cl. 514/252.11; 514/255.05; 544/405; 544/357

(57) ABSTRACT

The present invention relates to novel pyrazinylmethyl-lactam derivatives, that are compounds of the formula I 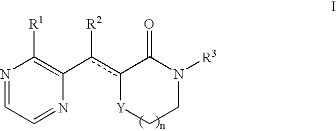
wherein R¹ is a group of the formula G¹ or G² depicted below, 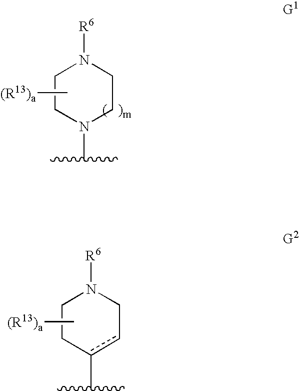
wherein R¹, R³, R⁶, R¹³ X, a, n and m are as defined herein, their pharmaceutically acceptable salts, and pharmaceutical compositions which include selective agonists, antagonists, inverse agonists and partial agonists of serotonin 1 (5-HT₁) receptors, specifically, of one or both of the 5-HT_{1A} and 5-HT_{1B} receptors. The compounds of the invention are useful in treating or preventing depression, anxiety, obsessive compulsive disorder (OCD) and other disorders for which a 5-HT₁ agonist or antagonist is indicated.

The present invention relates to novel pyrazinylmethyl lactam derivatives, to intermediates for their preparation, to pharmaceutical compositions containing them and to their medicinal use. The compounds of the present invention include selective agonists, antagonists, inverse agonists and partial agonists of serotonin 1 (5-HT₁) receptors, specifically, of one or both of the 5-HT_{1A} and 5-HT_{1B} (formerly classified 5-HT_{1D}) receptors. They are useful in treating or preventing depression, anxiety, obsessive compulsive disorder (OCD) and other disorders for which a 5-HT₁ agonist or antagonist is indicated.

European Patent Publication 434,561, published Jun. 26, 1991, refers to 7-alkyl alkoxy, and hydroxy substituted-1-(4-substituted-1-piperazinyl)-naphthalenes. The compounds are referred to as 5-HT₁ agonists and antagonists useful for the treatment of migraine, depression, anxiety, schizophrenia, stress and pain.

European Patent Publication 343,050, published Nov. 23, 1989, refers to 7-unsubstituted, halogenated, and methoxy substituted-1-(4-substituted-1-piperazinyl)-naphthalenes as useful 5-HT.sub.1A ligand therapeutics.

PCT Publication WO94/21619, published Sep. 29, 1994, refers to naphthalene derivatives as 5-HT₁ agonists and antagonists.

PCT Publication WO96/00720, published Jan. 11, 1996, refers to naphthyl ethers as useful 5-HT₁ agonists and antagonists.

European Patent Publication 701,819, published Mar. 20, 1996, refers to the use of 5-HT₁ agonists and antagonists in combination with a 5-HT re-uptake inhibitor.

Glennon et al. refers to 7-methoxy-1-(1-piperazinyl)-naphthalene as a useful 5-HT₁ ligand in their article “5-HT_{1D} Serotonin Receptors”, Clinical Drug Res. Dev., 22, 25-36 (1991).

Glennon's article “Serotonin Receptors: Clinical Implications”, Neuroscience and Behavioral Reviews, 14, 35-47 (1990), refers to the pharmacological effects associated with serotonin receptors including appetite suppression, thermoregulation, cardiovascular/hypotensive effects, sleep, psychosis, anxiety, depression, nausea, emesis, Alzheimer's disease, Parkinson's disease and Huntington's disease.

PCT Publication WO 95/31988, published Nov. 30, 1995, refers to the use of a 5-HT_{1D} antagonist in combination with a 5-HT_{1A} antagonist to treat CNS disorders such as depression, generalized anxiety, panic disorder, agoraphobia, social phobias, obsessive-compulsive disorder, post-traumatic stress disorder, memory disorders, anorexia nervosa and bulimia nervosa, Parkinson's disease, tardive dyskinesias, endocrine disorders such as hyperprolactinaemia, vasospasm (particularly in the cerebral vasculature) and hypertension, disorders of the gastrointestinal tract where changes in motility and secretion are involved, as well as sexual dysfunction.

G. Maura et al. J. Neurochem, 66, 203-209 (1996), have stated that administration of agonists selective for 5-HT_{1A} receptors or for both 5-HT_{1A} and 5-HT_{1D} receptors might represent a great improvement in the treatment of human cerebellar ataxias, a multifaceted syndrome for which no established therapy is available.

European Patent Publication 666,261, published Aug. 9, 1995, refers to thiazine and thiomorpholine derivatives which are claimed to be useful for the treatment of cataracts.

### SUMMARY OF THE INVENTION

The present invention relates to pyrazinylmethyl lactams of the formula 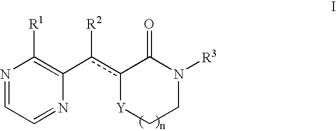
wherein R¹ is a group of the formula G¹ or G² depicted below, 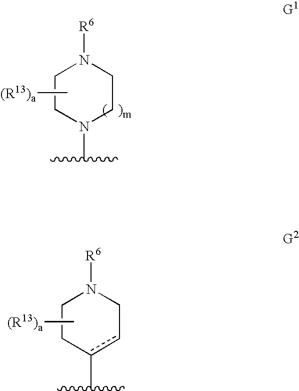
wherein R⁶ is hydrogen or —C(═O)—OR wherein R is C₁-C₈ straight chain or branched alkyl, C₃-C₈ cycloalkyl, or aryl; or

R⁶ is (C₁-C₆)alkyl or (C₁-C₄)alkyl-aryl wherein said aryl moiety is phenyl or naphthyl, optionally substituted with one or more substituents independently selected from (C₁-C₆)alkyl, (C₁-C₆)alkoxy, trifluoromethyl, cyano and SO_{g}(C₁-C₆)alkyl wherein g is zero, one or two;

each R¹³ is, independently, hydrogen, (C₁-C₄)alkyl, benzyl, or a (C₁-C₄)alkylene bridge from one of the ring carbons of the piperazine ring of G¹ to a ring carbon of the same ring or another ring or to a ring nitrogen of the piperazine ring having an available bonding site, or to a ring carbon of R⁶, when R⁶ has a ring structure having an available bonding site or a (C₁-C₄)alkylene bridge from one of the ring carbons of the piperidine ring of G² to a ring carbon of the same ring or another ring or to an amine substituent of the piperidine ring having an available bonding site, or to a ring carbon of R⁷ or R⁸, when either of R⁷ or R⁸ has a ring structure having an available bonding site;

a is zero to eight;

m is one, two or three;

Y is carbon, sulfur, nitrogen or oxygen;

R² is hydrogen, (C₁-C₆)alkyl, or benzyl;

R³ is vinyl, C(═O)R, wherein R is straight chain or branched (C₁-C₈)alkyl, (C₃-C₈)cycloalkyl, trifluoromethyl, or aryl; or,

R³ is —(CH₂)_{g}B, wherein g is zero to three and B is hydrogen, phenyl, naphthyl or a 5 to 7-membered heteroaryl ring containing from one to four heteroatoms in the ring selected from oxygen, nitrogen and sulfur, with the proviso that said ring cannot contain two adjacent oxygen atoms or two adjacent sulfur atoms and wherein the foregoing phenyl, naphthyl and heteroaryl rings may optionally be substituted with one to three substituents independently selected from chloro, fluoro, bromo, iodo, aryl-O—, heteroaryl-O—, aryl(C═O), heteroaryl(C═O), (C₁-C₈)alkyl, (C₁-C₈)hydroxyalkyl-, (C₁-C₈)alkoxy, (C₁-C₈)alkoxy-(C₁-C₈)alkyl-, (C₃-C₈)cycloalkyl-, (C₃-C₈)hydroxycycloalkyl, (C₃-C₈)cycloalkyl-O—, and wherein one to three carbon atoms of each of the foregoing (C₃-C₈)cycloalkyl substituents may be replaced with a heteroatom independently selected from nitrogen, oxygen or sulfur to form a heterocycloalkyl substituent having 4 to 8 atoms, with the proviso that said heterocycloalkyl substituent cannot contain two adjacent oxygen atoms or two adjacent sulfur atoms, and wherein each (C₃-C₈)cycloalkyl or heterocycloalkyl substituent may be independently substituted with from zero to three substituents independently selected from (C₁-C₈)alkyl, (C₁-C₄)alkyl-aryl wherein said aryl moiety is phenyl or naphthyl, hydroxy, and (C₁-C₈)alkoxy;

wherein when B is phenyl, naphthyl or heteroaryl, B may be optionally substituted with zero to three substituents independently selected from phenyl, naphthyl or a 5 to 7-membered heteroaryl ring containing from one to four heteroatoms selected from oxygen, nitrogen and sulfur, with the proviso that said heteroaryl ring cannot contain two adjacent oxygen atoms or two adjacent sulfur atoms, and wherein each independently selected phenyl, naphthyl or heteroaryl substituent may itself be independently substituted with from zero, one, two or three (C₁-C₈)alkyl or halo substituents; or, B may be optionally substituted with from zero to three substituents independently selected from nitro, trifluoromethyl, trifluoromethoxy, cyano, hydroxy, —CH₂OH, —COOH or the lactone formed from hydroxy or —CH₂OH with an ortho —COOH, and —SOₜ(C₁-C₆)alkyl wherein t is zero to two, or —CONR¹⁴R¹⁵, wherein R¹⁴ and R¹⁵ are independently selected from (C₁-C₈)alkyl, benzyl, or R¹⁴ and R¹⁵ together with the nitrogen to which they are attached form a 5 to 7-membered heteroalkyl ring that may contain from zero to three heteroatoms selected from nitrogen, sulfur and oxygen in addition to the nitrogen of the —CONR¹⁴R¹⁵ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms, or —(CH₂)ᵥNCOR¹⁶R¹⁷ wherein v is zero to three and —COR¹⁶ and R¹⁷ taken together with the nitrogen to which they are attached form a 4 to 6-membered lactam ring;

n is zero, one or two;

wherein the broken line indicates an optional double bond;

or, a pharmaceutically acceptable salt thereof.

Other embodiments of the invention relate to a compound according to formula I wherein R³ is (CH₂)_{g}B wherein g is zero and B is selected from phenyl and heteroaryl.

The invention also relates to a compound according to formula I wherein R³ is (CH₂)_{g}B wherein g is zero and B is selected from phenyl and heteroaryl wherein said phenyl or heteroaryl has one to three substituents independently selected from: (C₁-C₈)alkyl, (C₃-C₈)cycloalkyl-, (C₃-C₈)cycloalkyl-O—, wherein one to three carbon atoms of each of the foregoing (C₃-C₈)cycloalkyl substituents may be replaced with a heteroatom independently selected from nitrogen, oxygen and sulfur to form a heterocycloalkyl substituent having 4 to 8 atoms, with the proviso that said heterocycloalkyl substituent cannot contain two adjacent oxygen atoms or two adjacent sulfur atoms, and wherein each (C₃-C₈)cycloalkyl or heterocycloalkyl substituent may be independently substituted with from zero to three substituents independently selected from (C₁-C₈)alkyl, (C₁-C₄)alkyl-aryl, hydroxy, and (C₁-C₈)alkoxy, wherein said aryl moiety is phenyl or naphthyl.

The invention also relates to a compound according to formula I wherein R³ is (CH₂)_{g}B wherein g is zero and B is selected from phenyl and heteroaryl, wherein said phenyl or heteroaryl has one to three substituents independently selected from: tetrahydropyranyl, morpholinyl, azetidinyl, pyrrolidinyl, piperidyl, piperazinyl, thiomorpholinyl, hexahydroazepinyl, diazepinyl, oxazepinyl, thiazepinyl, oxadiazepinyl, thiadiazepinyl or triazepinyl, oxetanyl, tetrahydrofuranyl and wherein each said substituent may be independently substituted with from zero to three substituents independently selected from (C₁-C₈)alkyl.

The invention also relates to a compound according to formula I wherein R³ is (CH₂)_{g}B wherein g is zero and B is selected from phenyl and heteroaryl, wherein said phenyl or heteroaryl is optionally substituted with one to three substituents independently selected from: pyridyl, pyrrolyl, pyrimidyl, pyrazinyl, pyridazinyl, pyrazolyl, imidazolyl, triazolyl, tetrazolyl, oxazolyl, isoxazolyl, oxadiazolyl, thienyl, thiazolyl, thiadiazolyl, and isothiazolyl.

The invention also relates to a compound according to formula I wherein Y is carbon or oxygen and n is zero or one.

The invention also relates to a compound according to formula I wherein R⁶ is selected from hydrogen, (C₁-C₆)alkyl and (C₁-C₄)alkyl-aryl wherein said aryl moiety is phenyl or naphthyl, and —C(═O)—O(C₁-C₈)alkyl, R¹³ is (C₁-C₈)alkyl, a is zero to three and m is one.

The invention also relates to a compound according to formula I wherein R⁶ is selected from hydrogen, methyl, ethyl and benzyl, R¹³ is methyl, a is zero, one or two, m is one and n is zero or one.

The invention also relates to a compound according to formula I wherein R³ is (CH₂)_{g}B wherein g is zero and B is selected from phenyl and pyridyl, wherein said R¹⁴ and said R¹⁵ groups of said —CONR¹⁴R¹⁵ substituent together with the nitrogen to which they are attached form a 5 to 7-membered heteroalkyl ring selected from piperidine, N—(C₀-C₆)alkylpiperazine and morpholine.

Specific non-limiting examples of B wherein B is a 5 to 7-membered heteroaryl ring include pyridyl, pyrrolyl, pyrimidyl, pyrazinyl, pyridazinyl, pyrazolyl, imidazolyl, triazolyl, tetrazolyl, oxazolyl, isoxazolyl, oxadiazolyl, thienyl, thiazolyl, thiadiazolyl, and isothiazolyl.

Specific non-limiting examples of heterocycloalkyl substituents when B is phenyl or a 5 to 7-membered heteroaryl ring include pyrrolidinyl, piperidyl, piperazinyl, morpholinyl, thiomorpholinyl, diazepinyl, oxazepinyl, thiazepinyl, oxadiazepinyl, thiadiazepinyl, triazepinyl, tetrahydropyranyl, azetidinyl, hexahydroazepinyl, oxetanyl and tetrahydrofuranyl.

Specific examples of the compounds of the present invention are as follows:

1-(4-tert-butyl-phenyl)-3-(4-methyl-3,4,5,6-tetrahydro-2H-[1,2′]bipyrazinyl-3′-ylmethyl)-pyrrolidin-2-one;

3-(4-Methyl-3,4,5,6-tetrahydro-2H-[1,2′]bipyrazinyl-3′-ylmethyl)-1-[4-(tetrahydro-pyran-4-yl)-phenyl]-pyrrolidin-2-one;

4-(4-tert-Butyl-phenyl)-2-(4-methyl-3,4,5,6-tetrahydro-2H-[1,2′]bipyrazinyl-3′-ylmethyl)-morpholin-3-one;

1-(4-tert-Butyl-phenyl)-3-(4-methyl-3,4,5,6-tetrahydro-2H-[1,2′]bipyrazinyl-3′-ylmethyl)-piperidin-2-one;

3-(4-Methyl-3,4,5,6-tetrahydro-2H-[1,2′]bipyrazinyl-3′-ylmethyl)-1-[4-(tetrahydro-pyran-4-yl)-phenyl]-piperidin-2-one;

3-(4-methyl-3,4,5,6-tetrahydro-2H-[1,2′]bipyrazinyl-3′-ylmethylene)-piperidin-2-one;

3-(4-methyl-3,4,5,6-tetrahydro-2H-[1,2′]bipyrazinyl-3′-ylmethyl)-piperidin-2-one;

3-(4-methyl-3,4,5,6-tetrahydro-2H-[1,2′]bipyrazinyl-3′-ylmethyl)-1-[4-(4-methyl-tetrahydro-pyran-4-yl)-phenyl]piperidin-2-one;

(+)-1-(4-tert-butyl-phenyl)-3-(4-methyl-3,4,5,6-tetrahydro-2H-[1,2′]bipyrazinyl-3′-ylmethyl)-pyrrolidin-2-one;

(+)-3-(4-Methyl-3,4,5,6-tetrahydro-2H-[1,2′]bipyrazinyl-3′-ylmethyl)-1-[4-(tetrahydro-pyran-4-yl)-phenyl]-pyrrolidin-2-one;

(+)-4-(4-tert-Butyl-phenyl)-2-(4-methyl-3,4,5,6-tetrahydro-2H-[1,2′]bipyrazinyl-3′-ylmethyl)-morpholin-3-one;

(+)-1-(4-tert-Butyl-phenyl)-3-(4-methyl-3,4,5,6-tetrahydro-2H-[1,2′]bipyrazinyl-3′-ylmethyl)-piperidin-2-one;

(+)-3-(4-Methyl-3,4,5,6-tetrahydro-2H-[1,2′]bipyrazinyl-3′-ylmethyl)-1-[4-(tetrahydro-pyran-4-yl)-phenyl]-piperidin-2-one;

(+)-3-(4-methyl-3,4,5,6-tetrahydro-2H-[1,2′]bipyrazinyl-3′-ylmethyl)-piperidin-2-one;

(+)-3-(4-methyl-3,4,5,6-tetrahydro-2H-[1,2′]bipyrazinyl-3′-ylmethyl)-1-[4-(4-methyl-tetrahydro-pyran-4-yl)-phenyl]piperidin-2-one;

(−)-1-(4-tert-butyl-phenyl)-3-(4-methyl-3,4,5,6-tetrahydro-2H-[1,2′]bipyrazinyl-3′-ylmethyl)-pyrrolidin-2-one;

(−)-3-(4-Methyl-3,4,5,6-tetrahydro-2H-[1,2′]bipyrazinyl-3′-ylmethyl)-1-[4-(tetrahydro-pyran-4-yl)-phenyl]-pyrrolidin-2-one;

(−)-4-(4-tert-Butyl-phenyl)-2-(4-methyl-3,4,5,6-tetrahydro-2H-[1,2′]bipyrazinyl-3′-ylmethyl)-morpholin-3-one;

(−)-1-(4-tert-Butyl-phenyl)-3-(4-methyl-3,4,5,6-tetrahydro-2H-[1,2′]bipyrazinyl-3′-ylmethyl)-piperidin-2-one;

(−)-3-(4-Methyl-3,4,5,6-tetrahydro-2H-[1,2′]bipyrazinyl-3′-ylmethyl)-1-[4-(tetrahydro-pyran-4-yl)-phenyl]-piperidin-2-one;

(−)-3-(4-Methyl-3,4,5,6-tetrahydro-2H-[1,2′]bipyrazinyl-3′-ylmethyl)-piperidin-2-one;

(−)-3-(4-Methyl-3,4,5,6-tetrahydro-2H-[1,2′]bipyrazinyl-3′-ylmethyl)-1-[4-(4-methyl-tetrahydro-pyran-4-yl)-phenyl]piperidin-2-one; and, pharmaceutically acceptable salts thereof.

Unless otherwise indicated, the term “halo” as used herein includes fluoro, chloro, bromo and iodo. Unless otherwise indicated, the term “alkyl” as used herein includes straight or branched alkyl. Unless otherwise indicated, the term “cycloalkyl” as used herein includes moieties derived from cyclic hydrocarbons which have a linkage from a ring carbon to another group and includes cyclic hydrocarbon moieties substituted with straight or branched alkyl moieties.

The term “alkoxy” as used herein means “alkyl-O—”, wherein “alkyl” is defined as above.

The term “cycloalkyl-O—” as used herein means “cycloalkyl” as defined above in which the cycloalkyl moiety is linked by a single bond to an oxygen atom with the oxygen atom having an available bonding site for formation of an ether linkage.

The term “alkylene” as used herein means an alkyl radical having two available bonding sites (i.e., -alkyl-), wherein “alkyl” is defined as above.

The term “alkenyl” is intended to include hydrocarbon chains of either a straight or branched configuration comprising one or more unsaturated carbon-carbon bonds which may occur in any stable point along the chain, such as ethenyl and propenyl. Alkenyl groups typically will have 2 to about 12 carbon atoms, more typically 2 to about 8 carbon atoms.

The term “alkynyl” is intended to include hydrocarbon chains of either a straight or branched configuration comprising one or more triple carbon-carbon bonds which may occur in any stable point along the chain, such as ethynyl and propynyl. Alkynyl groups typically will have 2 to about 12 carbon atoms, more typically 2 to about 8 carbon atoms.

The term “aryl” is intended to include groups that, in accordance with the theory of Hückel, have a cyclic, delocalized (4n+2) pi-electron system. Examples of aryl groups include, but are not limited to, arenes and their substitution products, e.g., phenyl, naphthyl and toluyl, among numerous others.

The term “heteroaryl” is intended to include aromatic heterocyclic groups and includes the non-limiting examples thiophenyl, pyridyl, pyrimidyl, pyridazyl, oxazolyl, isooxazolyl, thiazolyl and isothiazolyl, among others.

Unless otherwise indicated, the term “heterocycloalkyl” as used herein includes a cyclic hydrocarbon in which one or more of the ring carbon atoms has been replaced with a nitrogen, oxygen or sulfur atom or any combination thereof.

Unless otherwise indicated, the term “one or more substituents” as used herein refers to from one to the maximum number of substituents possible based on the number of available bonding sites.

The compounds of formula I may have chiral centers and therefore may occur in different enantiomeric configurations. The invention includes all enantiomers, diastereomers, and other stereoisomers of such compounds of formula I, as well as racemic and other mixtures thereof.

The present invention also relates to the pharmaceutically acceptable acid addition salts of the compounds of formula I. Examples of pharmaceutically acceptable acid addition salts of the compounds of formula I are the salts of hydrochloric acid, p-toluenesulfonic acid, fumaric acid, citric acid, succinic acid, salicylic acid, oxalic acid, hydrobromic acid, phosphoric acid, methanesulfonic acid, tartaric acid, malate, di-p-toluoyl tartaric acid, and mandelic acid.

The present invention also relates to all radiolabeled forms of the compounds of the formula I. Preferred radiolabeled compounds of formula I are those wherein the radiolabels are selected from as ³H, ¹¹C, ¹⁴C, ¹⁸F, ¹²³I and ¹²⁵I. Such radiolabeled compounds are useful as research and diagnostic tools in metabolism pharmacokinetics studies and in binding assays in both animals and man.

The present invention also relates to a pharmaceutical composition for treating a disorder or condition in a mammal, including a human, selected from depression, anxiety, depression with concomitant anxiety, post traumatic stress disorder, panic phobias, obsessive compulsive disorder (OCD), borderline personality disorder, sleep disorder, psychosis, seizures, dyskinesis, symptoms of Huntington's or Parkinson's diseases, spasticity, suppression of seizures resulting from epilepsy, cerebral ischemia, anorexia, faintness attacks, hypokinesia, cranial traumas, chemical dependencies, premature ejaculation, premenstrual syndrome (PMS) associated mood and appetite disorder, inflammatory bowel disease, modification of feeding behavior, blocking carbohydrate cravings, late luteal phase dysphoric disorder, tobacco withdrawal-associated symptoms, panic disorder, bipolar disorder, sleep disorders, jet lag, cognitive dysfunction, hypertension, bulimia, anorexia, obesity, cardiac arrhythmias, chemical dependencies and addictions (e.g., dependencies on, or addictions to nicotine (and/or tobacco products), alcohol, benzodiazepines, barbiturates, opioids or cocaine), headache, stroke, traumatic brain injury (TBI), psychosis, Huntington's Chorea, tardive dyskinesia, hyperkinesia, dyslexia, schizophrenia, multi-infarct dementia, epilepsy, senile dementia of the Alzheimer's type (AD), Parkinson's disease (PD), attention deficit hyperactivity disorder (ADHD) and Tourette's Syndrome, comprising an amount of a compound of the formula I, or a pharmaceutically acceptable salt thereof, and a pharmaceutically acceptable carrier.

The present invention also relates to a method of treating a disorder or condition in a mammal, including a human, selected from depression, anxiety, depression with concomitant anxiety, post traumatic stress disorder, panic phobias, obsessive compulsive disorder (OCD), borderline personality disorder, sleep disorder, psychosis, seizures, dyskinesis, symptoms of Huntington's or Parkinson's diseases, spasticity, suppression of seizures resulting from epilepsy, cerebral ischemia, anorexia, faintness attacks, hypokinesia, cranial traumas, chemical dependencies, premature ejaculation, premenstrual syndrome (PMS) associated mood and appetite disorder, inflammatory bowel disease, modification of feeding behavior, blocking carbohydrate cravings, late luteal phase dysphoric disorder, tobacco withdrawal-associated symptoms, panic disorder, bipolar disorder, sleep disorders, jet lag, cognitive dysfunction, hypertension, bulimia, anorexia, obesity, cardiac arrhythmias, chemical dependencies and addictions (e.g., dependencies on, or addictions to nicotine (and/or tobacco products), alcohol, benzodiazepines, barbiturates, opioids or cocaine), headache, stroke, traumatic brain injury (TBI), psychosis, Huntington's Chorea, tardive dyskinesia, hyperkinesia, dyslexia, schizophrenia, multi-infarct dementia, epilepsy, senile dementia of the Alzheimer's type (AD), Parkinson's disease (PD), attention deficit hyperactivity disorder (ADHD) and Tourette's Syndrome, comprising administering to a mammal in need of such treatment an amount of a compound of the formula I, or a pharmaceutically acceptable salt thereof, that is effective in treating such disorder or condition.

### DETAILED DESCRIPTION OF THE INVENTION

Except where otherwise stated, R, R¹, R³, R⁶, R⁷, R⁸, R¹³, G¹, G², a, m, n and Y in the reaction schemes and discussion that follow are defined as above. Unless otherwise stated reaction conditions include an inert atmosphere commonly used in the art such as nitrogen or argon.

Scheme 1 refers to methods for the preparation of compounds of formula I wherein R¹ is G¹.

In step 1 of Scheme 1, the aldehyde of formula III is prepared by treating a 2-halo-3-formylpyrazine, compound V wherein X is F, Cl, Br or I (prepared according to the methods of A. Turck, L. Mojovic, and G. Queguiner, in Synthesis, 1988, pages 881-884 and N. Pie, A. Turck, A. Heynderickx and G. Queguiner, in Tetrahedron, 1998, vol. 54, pages 4899 4912) with an amine corresponding to G¹ (compound IV) in a solvent such as water, a lower alcohol, acetonitrile, tetrahydrofuran, 1,4-dioxane or mixtures thereof, preferably 1,4-dioxane, in the absence or presence of a base such as a trialkylamine, an alkali metal carbonate or an alkali metal hydrogen carbonate, preferably potassium carbonate at a temperature of about 0° C. to about 150° C., preferably about 60° C. to 120° C., for about 30 min to 12 h, preferably for about 1.5 h.

In step 2 of Scheme 1, the compound of formula IA is prepared by condensation of the aldehyde of formula III with an N-substituted lactam of the formula II by (a) treating the lactam of formula II in a reaction inert solvent such as diethylether, tetrahydrofuran (THF) or dioxane, preferably THF, with about 2 equivalents of an alkali metal amide base, such as lithium, sodium or potassium bis(trimethylsilylamide) or lithium, sodium or potassium diisopropylamide, preferably sodium bis(trimethylsilylamide), at a temperature of about −78° C. to about 50° C., preferably about −20° C. to about 25° C., followed by aging for about 15 min to 3 h, preferably for about 30 min, (b) adding chlorodiethylphosphate at a temperature of about −78° C. to about 50° C., preferably about −20° C. to about 25° C. followed by aging for about 30 min to 6 h, preferably for about 1.5 h, and (c) addition of the compound III with stirring at room temperature for about 30 min to 24 h, preferably for about 3 h to 18 h.

Step 3 of Scheme 1 is a catalytic reduction of the carbon-carbon double bond of IA to produce a compound of the formula I. The reduction of this double bond may be effected with hydrogen gas (H₂) in a reaction inert solvent such as a lower alcohol, THF, dioxane or ethyl acetate, preferably methanol, in the presence of a noble metal catalyst on a solid support such as palladium on carbon (Pd/C), palladium on barium sulfate (Pd/BaSO₄) or platinum on carbon (Pt/C) preferably about 10% palladium on carbon, at a pressure of from about 10 psi to about 100 psi, preferably about 40 psi to about 60 psi, at a temperature of about 20° C. to about 70° C., preferably about 40° C. to about 60° C. while shaking the reaction mixture for about 2 h to 72 h, preferably for about 24 to 36 h.

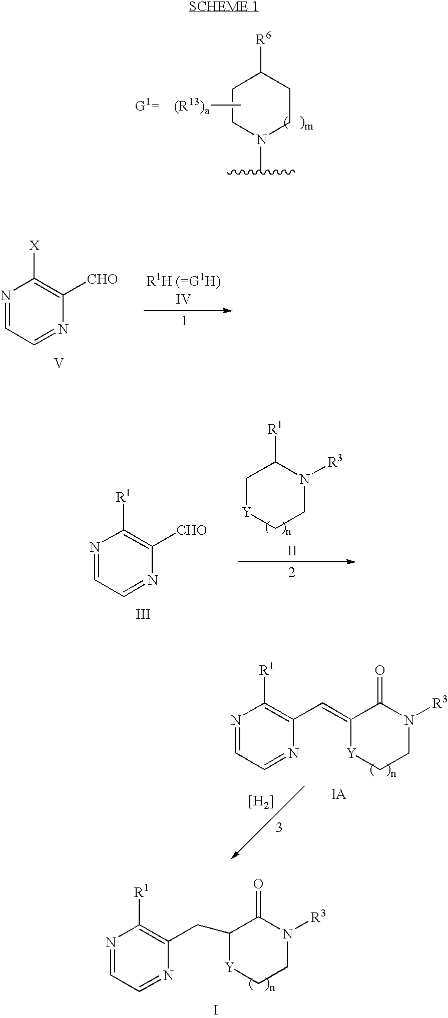

Scheme 1a refers to an alternative preparation of a compound of the formula I wherein Y is carbon, R¹ is G¹, n is zero, one or two and the optional double bond is either present or absent, beginning with substitution of the α-carbon atom of lactam VI with a reactive leaving group. Scheme 1a depicts the case wherein n=one.

In step 1 of Scheme 1a a lactam of the formula IX, wherein X is Cl, Br or I, preferably Br, is prepared by treating the lactam of formula VI with (a) a trialkylamine base, preferably diisopropylethylamine or triethylamine in a solvent such as dichloromethane or ethyl ether at a temperature of about 0° C. to about 40° C., preferably about 20° C. to about 25° C., followed by (b) the addition of a trialkylsilylsulfonate or trialkylsilylhalide preferably trimethylsilyltriflate, followed by stirring for about 20 min to 5 h, preferably for about 30 min to 2 h, then cooling to from about −90° C. to about 0° C., preferably from about −65°0 C. to about −75° C., followed by (c) the addition of a solution of bromine in dichloromethane that was then allowed to stir for about 20 min to 24 h, preferably for about 15 h.

In step 2 of Scheme 1a the lactam of formula X, wherein L is a dialkylphosphonate is prepared by treating the lactam of formula IX with a trialkylphosphite, preferably trimethylphosphite, in a solvent such as THF, 1,4-dioxane, propionitrile or butyronitrile, where butyronitrile is preferred, at a temperature of about 50° C. to about the reflux temperature of the solvent for about 3 h to 96 h, preferably for about 72 h.

In step 3 of Scheme 1a the compound of the formula IA is prepared by (a) treating a solution of the compound of formula X in a solvent such as ethyl ether, dioxane or tetrahydrofuran, preferably tetrahydrofuran, at a temperature of about 0° C. to about 40° C., with an alkali metal amide base, such as lithium, sodium or potassium bis(trimethylsilylamide) or lithium, sodium or potassium diisopropylamide, preferably sodium bis(trimethylsilylamide) followed by stirring for about 10 min to 3 h, preferably for about 30 min, followed by (b) addition of the compound of formula III with subsequent aging of about 5 min to 3 h, preferably for about 30 min.

Step 4 of Scheme 1a is a catalytic reduction of the carbon-carbon double bond of IA to produce a compound of the formula I. The reduction of this double bond may be effected with hydrogen gas (H₂) in a reaction inert solvent such as a lower alcohol, THF, dioxane or ethyl acetate, preferably methanol, in the presence of a noble metal catalyst on a solid support such as palladium on carbon (Pd/C), palladium on barium sulfate (Pd/BaSO₄) or platinum on carbon (Pt/C) preferably about 10% palladium on carbon, at a pressure of from about 10 psi to about 100 psi, preferably about 40 psi to about 60 psi, at a temperature of about 20° C. to about 70° C., preferably about 40° C. to about 60° C. while shaking the reaction mixture for about 2 h to 72 h, preferably for about 24 to 36 h.

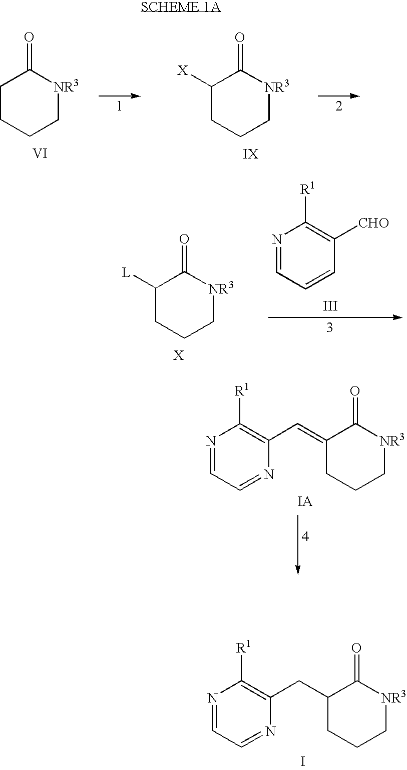

Scheme 1b refers to an another alternative preparation of a compound of the formula I wherein Y is carbon, R¹ is G¹, n is the integer zero or one, and the optional double bond is either present or absent, beginning with a compound of the formula XI, wherein Alk is lower alkyl, preferably ethyl (see Y. Shen and Z. Zhang, J. Chem. Res. Synop., 1999, 9, 556-557, for the preparation of compound XI). Scheme 1b depicts the case wherein n is one.

In step 1 of Scheme 1b the compound of formula XII can be prepared by treating an ethanolic solution of the compound of formula XI with Raney nickel under a hydrogen atmosphere of about 40 psi to about 50 psi, at a temperature of about 40° C. to about 60° C. in the presence of ammonium hydroxide for about 5 h to 48 h, preferably for about 24 h.

In step 2 of Scheme 1b the compound of formula IA′ can be prepared by (a) treating a solution of the compound of formula XII in a solvent such as ethyl ether, dioxane or tetrahydrofuran, preferably tetrahydrofuran, at a temperature of from about −40° C. to about 40° C., preferably from about −5° C. to about 5° C., with an alkali metal amide base, such as lithium, sodium or potassium bis(trimethylsilylamide) or lithium, sodium or potassium diisopropylamide, preferably sodium bis(trimethylsilylamide) with stirring for about 10 min to 3 h, preferably for about 30 min, followed by (b) addition of the compound of formula III with subsequent aging of about 5 min to 3 h, preferably for about 30 min.

In step 3 of Scheme 1b the compound of formula IA, wherein R³ is an optionally substituted aryl or heteroaryl group, can be prepared by treating a mixture of the compound of formula IA′, an aryl or heteroaryl chloride, bromide, iodide or sulfonate, preferably the bromide, a base such as potassium phosphate, potassium carbonate, sodium carbonate, thallium carbonate, cesium carbonate, potassium tert-butoxide, lithium tert-butoxide, or sodium tert-butoxide, preferably potassium carbonate, a diamine, such as 1,2-ethylenediamine, N,N′-dimethylethylenediamine, or cis-1,2-diaminocyclohexane, preferably N,N′-dimethyl-ethylenediamine, and cuprous chloride, bromide or iodide, preferably cuprous iodide, in the presence of a small amount of water, preferably about 1% to about 4% water, in a reaction inert solvent such as 1,2-dimethoxyethane, diglyme, t-butyl methyl ether, tetrahydrofuran, benzene or toluene, preferably toluene, at a temperature of from about 40° C. to about 150° C., preferably from about 80° C. to about 120° C. for about 15 h to 48 h, preferably for about 24 h.

The N-arylation or N-heteroarylation of step 3 to prepare the compound of formula IA, wherein R³ is an optionally substituted aryl or heteroaryl group, may also be accomplished by treating the compound of formula IA′ with an aryl or heteroaryl chloride, bromide, iodide, or sulfonate, preferably the bromide, a base such as an alkali metal carbonate, an alkali metal amine base, an alkali metal phosphonate, or an alkali metal alkoxide, preferably cesium carbonate, a phoshpine ligand, preferably 9,9-dimethyl-4,5-bis(diphenylphosphino)xanthene (XANTPHOS), and a palladium species, such as palladium (II) acetate or tris(dibenzylideneacetone)dipalladium(0) or the corresponding chloroform adduct, preferably tris(dibenzylideneacetone)dipalladium(0), in an inert solvent such as 1,4-dioxane or toluene, preferably 1,4-dioxane, at a temperature of from about 40° C. to about 160° C., preferably from about 80° C. to about 120° C., for about 15 h to 48 h, preferably for about 24 h.

In step 4 of Scheme 1b the compound of formula I, wherein R³ is an optionally substituted aryl or heteroaryl group, can be prepared from the compound of formula IA by the catalytic hydrogenation procedure of step 4 of Scheme 1a.

Alternatively, as depicted in step 3a of Scheme 1b the compound of formula IB can be prepared by catalytic hydrogenation of the compound of formula IA′ using the catalytic hydrogenation procedure of step 4 of Scheme 1a.

In step 4a of Scheme 1b the compound of formula I wherein R³ is an optionally substituted aryl or heteroaryl group, can be prepared from the compound of formula IB (the scheme has 1, not “1B”) by using the N-arylation and N-heteroarylation procedures of step 3 above.

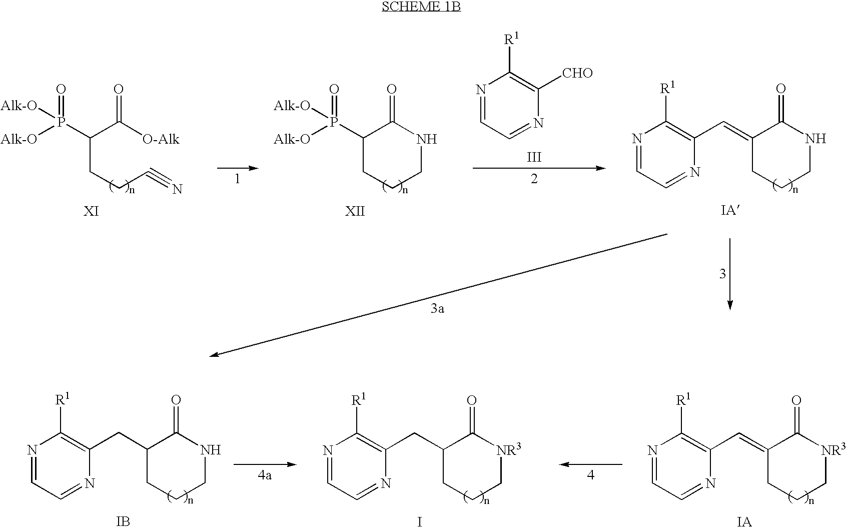

Scheme 2 refers to methods for the preparation of compounds of formula I wherein Y is oxygen, R¹ is G¹ and n is the integer one. In step 1 of Scheme 2 a lactam of the formula VII is prepared by halogenating, preferably brominating, a lactam of the formula VIa by standard means known in the art, preferably by means of N-bromosuccinimide in a solvent such as 1,2-dichloroethane at a temperature of about 60° C. to about 100° C. for about 5 h.

In step 2 of Scheme 2 the lactam of formula VIII, wherein the halogen atom, preferably the bromine atom of lactam VII, is replaced with a dialkylphosphonate group, is prepared by treating lactam VII with a trialkylphosphite, preferably trimethylphosphite, in a solvent such as 1,4-dioxane, acetonitrile, chloroform or THF, where THF is preferred at a temperature of about 50° C. to about the reflux temperature of the solvent for about 1 h to 24 h, where about 12 h to 18 h is preferred.

In step 3 of Scheme 2 the compound of formula IC can be prepared by (a) treating a solution of the compound of formula VIII in a solvent such as ethyl ether, dioxane or tetrahydrofuran, preferably tetrahydrofuran, at a temperature of about 0° C. to about 40° C., with an alkali metal amide base, such as lithium, sodium or potassium bis(trimethylsilylamide) or lithium, sodium or potassium diisopropylamide, preferably sodium bis(trimethylsilylamide) with stirring for about 10 min to 3 h, preferably for about 30 min, followed by (b) addition of the compound of formula III with subsequent aging of about 5 min to 3 h, preferably for about 30 min.

Step 4 of Scheme 2 is a catalytic reduction of the carbon-carbon double bond of IC to produce a compound of the formula ID. The reduction of this double bond may be effected with hydrogen gas (H₂) in a reaction inert solvent such as a lower alcohol, THF, dioxane or ethyl acetate, preferably methanol, in the presence of a noble metal catalyst on a solid support such as palladium on carbon (Pd/C), palladium on barium sulfate (Pd/BaSO₄) or platinum on carbon (Pt/C) preferably about 10% palladium on carbon, at a pressure of from about 10 psi to about 100 psi, preferably about 40 psi to about 60 psi, at a temperature of from about 20° C. to about 70° C., preferably from about 40° C. to about 60° C., while shaking the reaction mixture for about 2 h to 72 h, preferably for about 24 to 36 h.

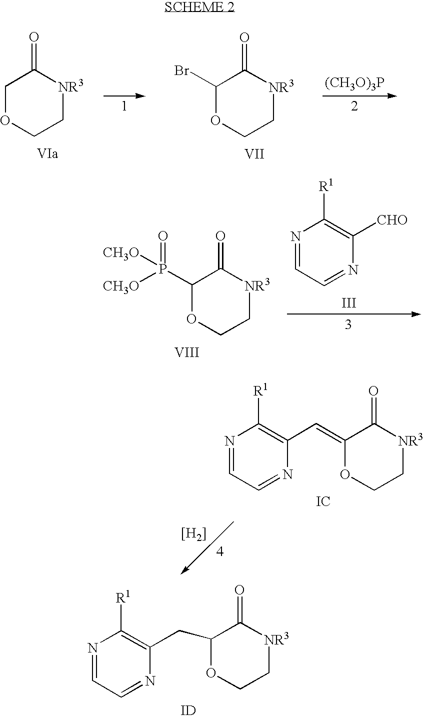

Scheme 3 refers to methods for the preparation of compounds of formula VI wherein Y is oxygen or carbon and R³ is an optionally substituted aryl or heteroaryl group as described above.

Compound VI can be prepared by treating a mixture of the compound of formula VIa, an aryl or heteroaryl chloride, bromide, iodide or sulfonate preferably the bromide, a base such as potassium phosphate, potassium carbonate, sodium carbonate, thallium carbonate, cesium carbonate, potassium tert-butoxide, lithium tert-butoxide, or sodium tert-butoxide, preferably, potassium carbonate, a diamine, such as 1,2-ethylenediamine, N,N′-dimethylethylenediamine, or cis-1,2-diaminocyclohexane, preferably N,N′-dimethylethylenediamine, and cuprous chloride, bromide or iodide, preferably cuprous iodide, in the presence of a small amount of water, preferably about 1% to about 4% water, in a reaction inert solvent such as 1,2-dimethoxyethane, diglyme, t-butyl methyl ether, tetrahydrofuran, benzene or toluene, preferably toluene, at a temperature of from about 40° C. to about 150° C., preferably from about 80° C. to about 120° C. for about 15 h to 48 h, preferably for about 24 h.

The N-arylation or N-heteroarylation to prepare the compound of formula VI, wherein R³ is an optionally substituted aryl or heteroaryl group, may also be accomplished by treating the compound of formula VIa with an aryl or heteroaryl chloride, bromide, iodide, or sulfonate, preferably the bromide, a base such as an alkali metal carbonate, an alkali metal amine base, an alkali metal phosphonate, or an alkali metal alkoxide, preferably cesium carbonate, a phoshpine ligand, preferably 9,9-dimethyl-4,5-bis(diphenylphosphino)xanthene (XANTPHOS), and a palladium species, such as palladium (II) acetate or tris(dibenzylideneacetone)dipalladium(0) or the corresponding chloroform adduct, preferably tris(dibenzylideneacetone)dipalladium(0), in an inert solvent such as 1,4-dioxane or toluene, preferably 1,4-dioxane, at a temperature of from about 40° C. to about 160° C., preferably from about 80° C. to about 120° C., for about 15 h to 48 h, preferably for about 24 h.

Compounds of formula VI wherein R³ is —(CH₂)_{g}B, wherein g is not zero and B is not aryl or heteroaryl can be prepared by alkylation of compounds of formula VIa by (i) treating compound VIa with a strong base/polar solvent system such as NaH/THF, NaH/DMF, or n-butyllithium/THF, at a temperature of from about −30° C. to about the reflux temperature of the solvent, for a period of about 5 minutes to about 24 hours and (ii) treating the anion thus formed with an alkylating agent of the formula R³A wherein A is F, Br, Cl, I or an alkyl or aryl sulfonate, for a period of about 5 min to 24 h.

Compounds of formula VI can also be prepared by condensation of a compound of formula XIII, wherein the group L¹ is halo, O(C₁-C₄)alkyl, hydroxy, or an activated carboxylic acid group derived from reaction of the corresponding carboxylic acid with a standard carboxylic acid activating reagent such as, but not limited to, a carbodiimide such as dicyclohexyl carbodiimide, 1-(3-dimethylaminopropyl)-3-ethylcarbodiimide hydrochloride salt) or a tripropylphosphonic anhydride, preferably Cl, and wherein the group L² is halo or an alkyl or aryl sulfonate preferably Cl, with a compound of the formula R³NH₂, wherein R³ is an optionally substituted aryl or heteroaryl group, (C₁-C₈)alkyl, or Ar(C₁-C₃)alkyl, wherein Ar is an optionally substituted aryl or heteroaryl group, in a solvent such as water, acetonitrile, 1,4 dioxane or tetrahydrofuran (THF), preferably THF, at a temperature of from about 10° C. to about 120° C., preferably 50° C. to about 80° C., in the presence or absence of a base such as triethylamine, diisopropylethyl amine, an alkali metal hydroxide or an alkali metal carbonate, preferably cesium carbonate, for a period of about 5 min to 24 h.

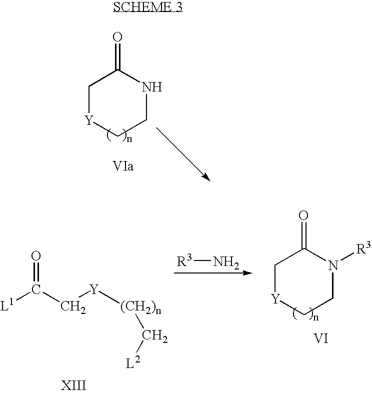

Scheme 4 refers to methods for the preparation of compounds of formula I wherein R¹ is G² and n is zero, one or two. The methods of Scheme 4 are analogous to the methods described in PCT Publication WO97/39867, the contents of which are incorporated herein by reference.

In step 1 of Scheme 4 a protected aldehyde is formed from the aldehyde of formula V wherein X is F, Cl, Br or I, using methods well known in the art such as preparation of the 1,3-dioxalane derivative by the method of J. E. Cole et al. (J. Chem. Soc., 1962, pp 244), by refluxing a solution of the aldehyde of formula V and 1,3-propanediol in anhydrous benzene with a catalytic amount of p-toluenesulfonic acid. Examples of other protecting groups may be found in T. W. Greene, Protecting Groups in Organic Synthesis, John Wiley & Sons, New York, 1981. Protective groups that are resistant to catalytic hydrogenation (e.g., 1,3-dioxolane) and which therefore allow for subsequent reduction, if required, of the carbon-carbon double bond of the tetrahydropyridines of formula XVII to yield compounds of formula XVIII wherein the optional double bond (depicted by the dotted line) is absent are most preferred.

In step 2 of Scheme 4 a compound of formula XVII is prepared by treating a protected aldehyde of the formula XV with vinyl stannanes of the formula XVI, wherein the Alk group is (C₁-C₆)alkyl, in the presence of a catalyst, preferably a Pd catalyst selected from (Ph₃P)₄Pd or Pd₂(dba)₃, wherein dba is dibenzylideneacetone. This reaction may be carried out as described in “Palladium-catalyzed Vinylation of Organic Halides” in Organic Reactions, Vol 27, pp. 345-390, W. B. Dauben, Ed., John Wiley & Sons, Inc., New York, N.Y., 1982.

In step 3a of Scheme 4 a compound of formula XVIIA, wherein the optional double bond is absent, is prepared from the compound of formula XVII by catalytic reduction of the double-bond using a noble metal catalyst such as palladium or platinum adsorbed onto carbon, preferably palladium on carbon, under a hydrogen atmosphere from about 10 psi to about 100 psi, preferably about 50 psi, in a solvent such as ethyl acetate, tetrahydrofuran, methanol, or ethanol, preferably methanol, at a temperature of from about 20° C. to about 70° C.

In step 3 or 3b, of Scheme 4 the deprotected aldehyde of formula XVIII is generated from either compound XVII or XVIIA using one or more of the techniques known in the art and described in the aforementioned publication by Greene, for example, by treating with an acid such as HCl having a concentration of about 5%, in a solvent such as THF at about room temperature for a period of about 5 min to 24 h.

In step 4, of Scheme 4 a compound of formula XIX can be prepared by means of the methods and procedures described in Scheme 1, step 2, Scheme 1A, step 3, Scheme 1B, step 2 and Scheme 2, step 3.

In step 5 of Scheme 4 a compound of the formula XX can be prepared by reduction of the double bond(s) of compound XIX using a noble metal catalyst such as palladium or platinum adsorbed onto carbon, preferably palladium on carbon, under a hydrogen atmosphere of about 10 to about 100 psi, preferably about 50 psi, in a solvent such as ethyl acetate, tetrahydrofuran, methanol, or ethanol, preferably methanol, at a temperature of from about 20° C. to about 70° C. for a period of about 2 h to 48 h.

In step 6 of Scheme 4, a compound of formula I, wherein R⁶ is hydrogen, can be prepared by removal of the protecting group on the piperidine nitrogen, exemplified by tert-butyoxycarbonyl in Scheme 4, using techniques known in the art and described in the aforementioned publication by Greene, for example, by treating compound XX with an acid such as about 3M hydrochloric acid in a solvent such as ethylacetate at about room temperature for a period of about 1 h to 24 h.

In step 7 of Scheme 4, a compound of formula I, wherein R⁶ is functionalized, can be prepared by reductively aminating a compound of formula I wherein R⁶ is hydrogen with an appropriate aldehyde or ketone in a solvent such as acetonitrile or methanol, a catalyst such as acetic acid, and a reducing agent such as sodium cyanoborohydride or sodium triacetoxyborohydride at about room temperature, or via alkylation with an alkyl halide or sulfonate in a solvent such as acetonitrile in the presence of a base such as sodium carbonate and a catalyst such as sodium iodide for a period of about 1 h to 24 h.

In step 8 of Scheme 4, a compound of the formula IA, wherein R⁶ is hydrogen, can be prepared by removal of the protecting group on the piperidine nitrogen, exemplified by tert-butyoxycarbonyl in Scheme 4, using techniques known in the art and described in the aforementioned publication by Greene, for example, by treating compound XIX with an acid such as about 3M hydrochloric acid in a solvent such as ethylacetate at about room temperature for a period of about 1 h to 24 h.

In step 11 of Scheme 4 a compound of the formula I can be prepared by reduction of the double bond(s) of compound IA using a noble metal catalyst such as palladium or platinum adsorbed onto carbon, preferably palladium on carbon, under a hydrogen atmosphere of about 10 to about 100 psi, preferably about 50 psi, in a solvent such as ethyl acetate, tetrahydrofuran, methanol, or ethanol, preferably methanol, at a temperature of from about 20° C. to about 70° C. for a period of about 2 h to 48 h. The secondary nitrogen of compound I wherein R⁶ is hydrogen can then be functionalized as described in step 7 of Scheme 4.

Yet another route to the compound of formula I wherein R⁶ is functionalized is depicted in Scheme 4, step 9, wherein the compound of formula IA, wherein R⁶ is hydrogen is treated as described in step 7, and in step 10, wherein the compound resulting from step 9 is catalytically reduced as described in step 11.

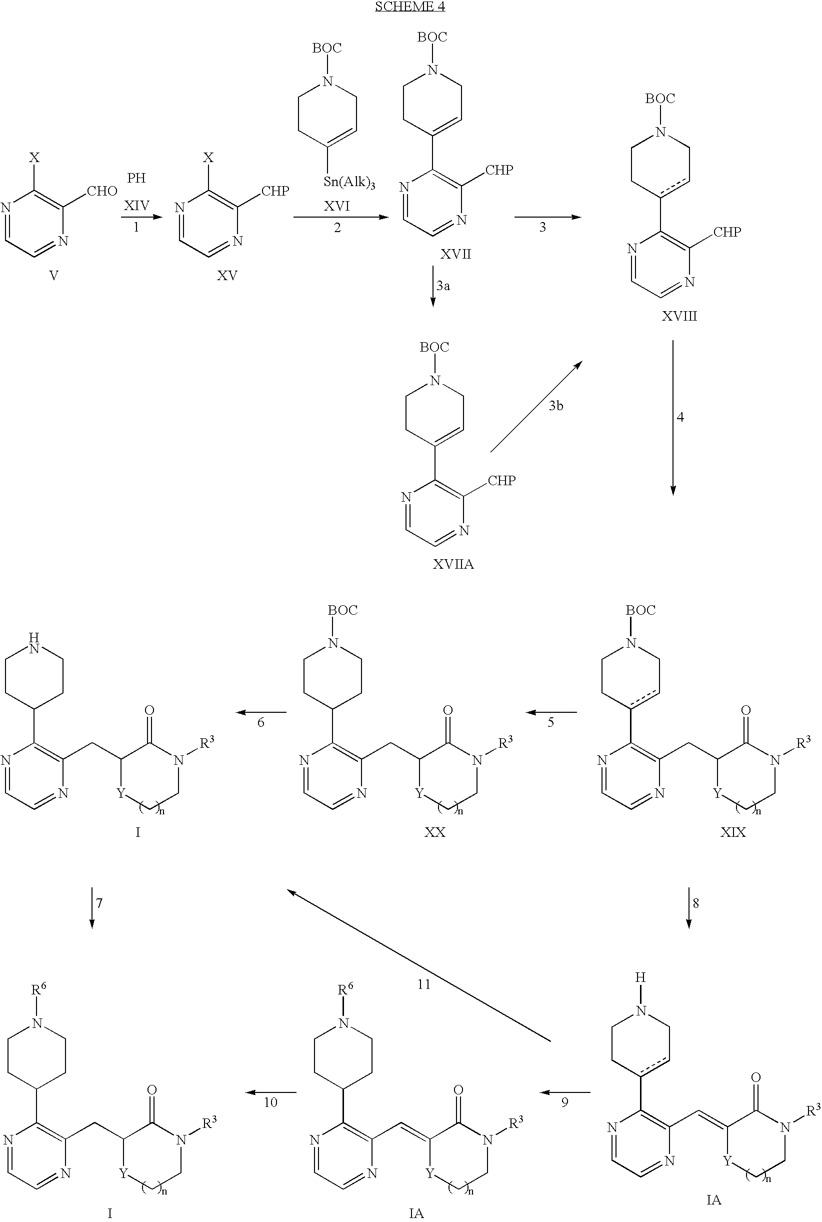

Aryl halides used in the N-arylation and N-heteroarylation coupling reactions described herein were either commercially available or could be prepared via the general methods given in U.S. Pat. No. 5,612,359; Guay, D., et al. Biorg. Med. Chem. Lett. 2002, 12, 1457-1461; Sall, D. J., et al. J. Med. Chem. 2000, 43, 649-663; Olah, G. A.; Porter, R. D., J. Amer. Chem. Soc. 1971, 93, 6877-6887; Brown, H. C., et al. J. Amer. Chem. Soc. 1957, 79, 1906-1909; Nenitzescu, C.; Necsoiu, I. J. Amer. Chem. Soc. 1950, 72, 3483-3486; Muci, A. R.; Buchwald, S. L., Top. Curr. Chem. 2002, 219, 131-209; DE 19650708; EP 104860; Wang, X., et al. Tetrahedron Lett., 2000, 41, 4335-4338. The contents of all of the foregoing are incorporated herein by reference in their entirety. Those skilled in the art will recognize that, where appropriate, hydroxyl groups on aryl or heteroaryl halides can be etherified by standard methods known in the art such as treatment with an alkali metal hydride or alkali metal hydroxide, such as sodium hydride, potassium hydride, sodium hydroxide, potassium hydroxide, or cesium hydroxide, preferably sodium hydride, in a solvent such as tetrahydrofuran, N,N-dimethylformamide, or dimethylsulfoxide, preferably tetrahydrofuran, at a temperature from about −20° C. to about 50° C., followed by addition of an alkyl halide or tosylate, preferably an alkyl iodide.

If the aryl or heteroaryl halide contains a benzylic hydroxyl, the hydroxyl can be reductively removed by treating the aryl or heteroaryl halide with a trialkyl or triarylsilane, preferably triethylsilane in a solvent such as methylene chloride, in the presence of an acid such as trifluoroacetic acid at a temperature of from about 0° C. to about 70° C., preferably about 25° C., for a period of about 5 min to 24 h.

Alternately, if the aryl or heteroaryl halide contains a benzylic hydroxyl, the hydroxyl can be converted to a methyl group using dichlorodimethyltitanium according to processes and procedures disclosed in the following publications: a) Reetz, M. T., Westerman, J., Kyung, S. H., Chem. Ber. 1985, 118, 1050-1057; b) Poon, T., et al. Synthesis, 1998, 832-834; and c) Harrowven, D. C., Hannam, J. C. Tetrahedron Lett., 1998, 39, 9573-9574.

The compounds of the formula I and their pharmaceutically acceptable salts (hereafter “the active compounds”) can be administered via either the oral, transdermal (e.g., through the use of a patch), intranasal, sublingual, rectal, parenteral or topical routes. Transdermal and oral administration are preferred. These compounds are, most desirably, administered in dosages ranging from about 0.25 mg up to about 1500 mg per day, preferably from about 0.25 to about 300 mg per day in single or divided doses, although variations will necessarily occur depending upon the weight and condition of the subject being treated and the particular route of administration chosen. However, a dosage level that is in the range of about 0.01 mg to about 10 mg per kg of body weight per day is most desirably employed. Variations may nevertheless occur depending upon the weight and condition of the persons being treated and their individual responses to said medicament, as well as on the type of pharmaceutical formulation chosen and the time period and interval during which such administration is carried out. In some instances, dosage levels below the lower limit of the aforesaid range may be more than adequate, while in other cases still larger doses may be employed without causing any harmful side effects, provided that such larger doses are first divided into several small doses for administration throughout the day.

The active compounds can be administered alone or in combination with pharmaceutically acceptable carriers or diluents by any of the several routes previously indicated. More particularly, the active compounds can be administered in a wide variety of different dosage forms, e.g., they may be combined with various pharmaceutically acceptable inert carriers in the form of tablets, capsules, transdermal patches, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents. In addition, oral pharmaceutical compositions can be suitably sweetened and/or flavored. In general, the active compounds are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc can be used for tabletting purposes. Solid compositions of a similar type may also be employed as fillers in gelatin capsules; preferred materials in this connection also include lactose or milk sugar] as well as high molecular weight polyethylene glycols. When aqueous suspensions and/or elixirs are desired for oral administration the active ingredient may be combined with various sweetening or flavoring agents, coloring matter and, if so desired, emulsifying and/or suspending agents, together with such diluents as water, ethanol, propylene glycol, glycerin and various combinations thereof.

For parenteral administration, a solution of an active compound in either sesame or peanut oil or in aqueous propylene glycol can be employed. The aqueous solutions should be suitably buffered (preferably pH greater than 8), if necessary, and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art.

It is also possible to administer the active compounds topically and this can be done by way of creams, a patch, jellies, gels, pastes, ointments and the like, in accordance with standard pharmaceutical practice.

### Biological Assay

The activity of the compounds of the present invention with respect to 5HT_{1B} (formerly referred to as 5HT_{1D}) binding ability can be determined using standard radioligand binding assays as described in the literature. The 5-HT_{1A} affinity can be measured using the procedure of Hoyer et al. (Brain Res., 1986, 376, 85). The 5-HT_{1D} affinity can be measured using the procedure of Heuring and Peroutka (J. Neurosci., 1987, 7, 894).

The in vitro activity of the compounds of the present invention at the 5-HT_{1D} binding site may be determined according to the following procedure. Bovine caudate tissue is homogenized and suspended in 20 volumes of a buffer containing 50 mM TRIS.hydrochloride (tris[hydroxymethyl]aminomethane hydrochloride) at a pH of 7.7. The homogenate is then centrifuged at 45,000 G for 10 minutes. The supernatant is then discarded and the resulting pellet resuspended in approximately 20 volumes of 50 mM TRIS.hydrochloride buffer at pH 7.7. This suspension is then pre-incubated for 15 minutes at 37° C., after which the suspension is centrifuged again at 45,000 G for 10 minutes and the supernatant discarded. The resulting pellet (approximately 1 gram) is resuspended in 150 ml of a buffer of 15 mM TRIS.hydrochloride containing 0.01 percent ascorbic acid with a final pH of 7.7 and also containing 10 μM pargyline and 4 mM calcium chloride (CaCl₂). The suspension is kept on ice at least 30 minutes prior to use.

The inhibitor, control or vehicle is then incubated according to the following procedure. To 50 μl of a 20 percent dimethylsulfoxide (DMSO)/80 percent distilled water solution is added 200 μl of tritiated 5-hydroxytryptamine (2 nM) in a buffer of 50 mM TRIS.hydrochloride containing 0.01 percent ascorbic acid at pH 7.7 and also containing 10 μM pargyline and 4 μM calcium chloride, plus 100 nM of 8-hydroxy-DPAT (dipropylaminotetraline) and 100 nM of mesulergine. To this mixture is added 750 μl of bovine caudate tissue, and the resulting suspension is vortexed to ensure a homogenous suspension. The suspension is then incubated in a shaking water bath for 30 minutes at 25° C. After incubation is complete, the suspension is filtered using glass fiber filters (e.g., Whatman GF/B-filters™). The pellet is then washed three times with 4 ml of a buffer of 50 mM TRIS.hydrochloride at pH 7.7. The pellet is then placed in a scintillation vial with 5 ml of scintillation fluid (aquasol 2™) and allowed to sit overnight. The percent inhibition can be calculated for each dose of the compound. An IC₅₀ value can then be calculated from the percent inhibition values.

The activity of the compounds of the present invention for 5-HT_{1A} binding ability can be determined according to the following procedure. Rat brain cortex tissue is homogenized and divided into samples of 1 gram lots and diluted with 10 volumes of 0.32 M sucrose solution. The suspension is then centrifuged at 900 G for 10 minutes and the supernate separated and recentrifuged at 70,000 G for 15 minutes. The supernate is discarded and the pellet re-suspended in 10 volumes of 15 mM TRIS.hydrochloride at pH 7.5. The suspension is allowed to incubate for 15 minutes at 37° C. After pre-incubation is complete, the suspension is centrifuged at 70,000 G for 15 minutes and the supernate discarded. The resulting tissue pellet is resuspended in a buffer of 50 mM TRIS.hydrochloride at pH 7.7 containing 4 mM of calcium chloride and 0.01 percent ascorbic acid. The tissue is stored at −70° C. until ready for an experiment. The tissue can be thawed immediately prior to use, diluted with 10 μm pargyline and kept on ice.

The tissue is then incubated according to the following procedure. Fifty microliters of control, inhibitor, or vehicle (1 percent DMSO final concentration) is prepared at various dosages. To this solution is added 200 μl of tritiated DPAT at a concentration of 1.5 nM in a buffer of 50 mM TRIS.hydrochloride at pH 7.7 containing 4 mM calcium chloride, 0.01 percent ascorbic acid and pargyline. To this solution is then added 750 μl of tissue and the resulting suspension is vortexed to ensure homogeneity. The suspension is then incubated in a shaking water bath for 30 minutes at 37° C. The solution is then filtered, washed twice with 4 ml of 10 mM TRIS.hydrochloride at pH 7.5 containing 154 mM of sodium chloride. The percent inhibition is calculated for each dose of the compound, control or vehicle. IC₅₀ values are calculated from the percent inhibition values.

The agonist and antagonist activities of the compounds of the invention at 5-HT_{1A} and 5-HT_{1D} receptors can be determined using a single saturating concentration according to the following procedure. Male Hartley guinea pigs are decapitated and 5-HT_{1A} receptors are dissected out of the hippocampus, while 5-HT_{1D} receptors are obtained by slicing at 350 mM on a McIlwain tissue chopper and dissecting out the substantia nigra from the appropriate slices. The individual tissues are homogenized in 5 mM HEPES buffer containing 1 mM EGTA (pH 7.5) using a hand-held glass-Teflon® homogenizer and centrifuged at 35,000×g for 10 minutes at 4° C. The pellets are resuspended in 100 mM HEPES buffer containing 1 mM EGTA (pH 7.5) to a final protein concentration of 20 mg (hippocampus) or 5 mg (substantia nigra) of protein per tube. The following agents are added so that the reaction mix in each tube contained 2.0 mM MgCl₂, 0.5 mM ATP, 1.0 mM cAMP, 0.5 mM IBMX, 10 mM phosphocreatine, 0.31 mg/mL creatine phosphokinase, 100 μM GTP and 0.5-1 microcuries of [³²P]-ATP (30 Ci/mmol: NEG-003-New England Nuclear). Incubation is initiated by the addition of tissue to siliconized microfuge tubes (in triplicate) at 30° C. for 15 minutes. Each tube receives 20 μL tissue, 10 μL drug or buffer (at 10× final concentration), 10 μL 32 nM agonist or buffer (at 10× final concentration), 20 μL forskolin (3 μM final concentration) and 40 μL of the preceding reaction mix. Incubation is terminated by the addition of 100 μL 2% SDS, 1.3 mM CAMP, 45 mM ATP solution containing 40,000 dpm [³H]-cAMP (30 Ci/mmol: NET-275-New England Nuclear) to monitor the recovery of CAMP from the columns. The separation of [³²P]-ATP and [³²P]-cAMP is accomplished using the method of Salomon et al., Analytical Biochemistry, 1974, 58, 541-548. Radioactivity is quantified by liquid scintillation counting. Maximal inhibition is defined by 10 μM (R)-8-OH-DPAT for 5-HT_{1A} receptors, and 320 nM 5-HT for 5-HT_{1D} receptors. Percent inhibitions by the test compounds are then calculated in relation to the inhibitory effect of (R)-8-OH-DPAT for 5-HT_{1A} receptors or 5-HT for 5-HT_{1D} receptors. The reversal of agonist induced inhibition of forskolin-stimulated adenylate cyclase activity is calculated in relation to the 32 nM agonist effect.

The compounds of the invention can be tested in vivo for antagonism of 5-HT_{1D} agonist-induced hypothermia in guinea pigs according to the following procedure.

Male Hartley guinea pigs from Charles River, weighing 250-275 grams on arrival and 300-600 grams at testing, serve as subjects in the experiment. The guinea pigs are housed under standard laboratory conditions on a 7 a.m. to 7 p.m. lighting schedule for at least seven days prior to experimentation. Food and water are available ad libitum until the time of testing.

The compounds of the invention can be administered as solutions in a volume of 1 ml/kg. The vehicle used is varied depending on compound solubility. Test compounds are typically administered either sixty minutes orally (p.o.) or 0 minutes subcutaneously (s.c.) prior to a 5-HT_{1D} agonist, such as [3-(1-methylpyrrolidin-2-ylmethyl)-1H-indol-5-yl]-(3-nitropyridin-3-yl)-amine, which can be prepared as described in PCT Publication WO93/11106, published Jun. 10, 1993, the contents of which are incorporated herein by reference in its entirety, and which is administered at a dose of 5.6 mg/kg, s.c. Before a first temperature reading is taken, each guinea pig is placed in a clear plastic shoe box containing wood chips and a metal grid floor and allowed to acclimate to the surroundings for 30 minutes. Animals are then returned to the same shoe box after each temperature reading. Prior to each temperature measurement, each animal is firmly held with one hand for a 30-second period. A digital thermometer with a small animal probe is used for temperature measurements. The probe is made of semi-flexible nylon with an epoxy tip. The temperature probe is inserted 6 cm. into the rectum and held there for 30 seconds or until a stable recording is obtained. Temperatures are then recorded.

In p.o. screening experiments, a “pre-drug” baseline temperature reading is made at −90 minutes, the test compound is given at −60 minutes and an additional −30 minute reading is taken. The 5-HT_{1D} agonist is then administered at 0 minutes and temperatures are taken 30, 60, 120 and 240 minutes later. In subcutaneous screening experiments, a pre-drug baseline temperature reading is made at −30 minutes. The test compound and 5-HT_{1D} agonists are given concurrently and temperatures are taken at 30, 60, 120 and 240 minutes later.

Data are analyzed with two-way analysis of variants with repeated measures in Newman-Keuls post hoc analysis.

The active compounds of the invention can be evaluated as anti-migraine agents by testing the extent to which they mimic sumatriptan in contracting the dog isolated saphenous vein strip (P. P. A. Humphrey et al., Br. J. Pharmacol., 1988, 94, 1128). This effect can be blocked by methiothepin, a known serotonin antagonist. Sumatriptan is known to be useful in the treatment of migraine and produces a selective increase in carotid vascular resistance in the anesthetized dog. The pharmacological basis of sumatriptan efficacy has been discussed in W. Fenwick et al., Br. J. Pharmacol., 1989, 96, 83.

The serotonin 5-HT₁ agonist activity can be determined by the in vitro receptor binding assays, as described for the 5-HT_{1A} receptor using rat cortex as the receptor source and [³H]-8-OH-DPAT as the radioligand (D. Hoyer et al., Eur. J. Pharm., 1985, 118, 13) and as described for the 5-HT_{1D} receptor using bovine caudate as the receptor source and [³H]serotonin as the radioligand (R. E. Heuring and S. J. Peroutka, J. Neuroscience, 1987, 7, 894).

The following experimental preparations and examples illustrate, but do not limit the scope of, this invention.

### PREPARATION 1

### 4-Methyl-3,4,5,6-tetrahydro-2H-[1,2′]bipyrazinyl-3′-carbaldehyde

n-BuLi (56 mmol, 22.4 mL, 2.5 M in hexanes) was added to tetrahydrofuran (300 mL) cooled to −78° C. followed by the addition of 2,2-6,6-tetramethylpiperidine (52 mmol, 8.71 mL). The solution was removed from the cooling bath and stirred for 30 minutes and then cooled back to −78° C. 2-chloropyrazine (40 mmol, 3.65 mL) was added dropwise, and the solution turned a reddish-brown color. After stirring 30 minutes, methylformate (60 mmol, 3.7 mL) was added and the reaction mixture was stirred for 2.25 hrs at −78° C. Acetic acid (8 mL) was added and the mixture was warmed to 0° C., was washed 3 times with 1:1 brine-water, dried over sodium sulfate, and then concentrated in vacuo. The residue was dissolved in 1,4-dioxane (250 mL) and 1-methylpiperazine (60 mmol, 6.6 mL) and potassium carbonate solution (8.28 g in 60 mL of water) were added and the mixture was heated at 100° C. for 1.5 hours. After cooling to room temperature, the mixture was filtered through a Celite pad which was then washed with chloroform. The filtrate was concentrated in vacuo and purified by silica gel chromatography (100:1:1 chloroform-methanol-ammonium hydroxide) to yield 3.3 g (40% yield for two steps) of 4-methyl-3,4,5,6-tetrahydro-2H-[1,2′]bipyrazinyl-3′-carbaldehyde; ¹³C NMR (100 MHz, CDCl₃) d 191.7, 154.3, 145.3, 134.5, 133.2, 55.1, 48.7, 46.3; MS (AP/CI) 207.2 (M+H)+.

### EXAMPLE 1

### 1-(4-tert-butyl-phenyl)-3-(4-methyl-3,4,5,6-tetrahydro-2H-[1,2′]bipyrazinyl-3′-ylmethyl)-pyrrolidin-2-one

A flame-dried flask under a nitrogen atmosphere was charged with dry tetrahydrofuran (10 mL) and diisopropylamine (834 μL, 5.97 mmol) and was cooled to −78° C. n-Butyllithium (5.97 mmol, 2.38 mL, 2.5 M in hexanes) was added dropwise, the solution was warmed to 0° C. for 15 minutes and was then cooled back to −78° C. 1-(4-tert-Butylphenyl)-pyrrolidin-2-one (2.91 mmol, 632 mg) in tetrahydrofuran (2.5 mL) was added dropwise and the cooling bath was then removed. After 30 minutes, chlorodiethylphosphate (419 μL, 2.91 mmol) was added dropwise and the solution was stirred for 1.5 hours. 4-Methyl-3,4,5,6-tetrahydro-2H-[1,2′]bipyrazinyl-3′-carbaldehyde (600 mg, 2.91 mmol) in tetrahydrofuran (2.5 mL) was added dropwise and the solution was stirred for 18 hours. Methanol was added and the mixture was purified by silica gel chromatography to yield 692 mg of the cis and trans isomers (1 a); MS (AP/CI) 406.3 (M+H)+. These isomers were dissolved in methanol (20 mL), 10% palladium on carbon (300 mg) was added, the mixture was placed under 50 psi hydrogen and was shaken for 28 hours. The mixture was then filtered through Celite, the solvent was removed in vacuo and the residue was purified by silica gel chromatography (110:1:1 chloroform-methanol-ammonium hydroxide) to yield 468 mg of 1-(4-tert-butyl-phenyl)-3-(4-methyl-3,4,5,6-tetrahydro-2H-[1,2′]bipyrazinyl-3′-ylmethyl)-pyrrolidin-2-one; ¹³C NMR (100 MHz, CDCl₃) d 175.7, 158.0, 148.2, 147.6, 139.5, 137.2, 136.9, 125.9, 119.7, 55.3, 49.7, 47.0, 46.4, 42.2, 35.5, 34.6, 31.6, 25.6; MS (AP/CI) 408.4 (M+H)+. Enantiomers were separable chromatographically (92/8 heptane-ethanol; Chiralcel OD, 10 cm×25 cm; 275 ml/min; t1=about 21 min; t2=about 32 minutes).

### EXAMPLE 2

### 3-(4-Methyl-3,4,5,6-tetrahydro-2H-[1,2′]bipyrazinyl-3′-ylmethyl)-1-[4-(tetrahydro-pyran-4-yl)-phenyl]-pyrrolidin-2-one

3-(4-Methyl-3,4,5,6-tetrahydro-2H-[1,2′]bipyrazinyl-3′-ylmethyl)-1-[4-(tetrahydro-pyran-4-yl)-phenyl]-pyrrolidin-2-one was prepared in an analogous manner to Example 1 using 1-[4-(tetrahydro-pyran-4-yl)-phenyl]-pyrrolidin-2-one; ¹³C NMR (100 MHz, CDCl₃) d 175.8, 158.0, 148.1, 142.3, 139.5, 138.0, 136.9, 127.3, 120.2, 68.6, 55.3, 49.7, 47.0, 46.4, 42.2, 41.2, 35.4, 34.2, 25.6; MS (AP/CI) 436.3 (M+H)+.

### General Procedure B

A phosphonate corresponding to formula VIII of Scheme 2, (10 mmole) dissolved in 10 mL of tetrahydrofuran cooled to 0° C. was treated dropwise with 1.1 equivalents of sodium bis(trimethylsilyl)amide (1M in tetrahydrofuran). After stirring for 30 minutes, a solution of 1.1 equivalents of an aldehyde corresponding to formula III of Scheme 1 or 2, in 2 mL of tetrahydrofuran was added and the solution was allowed to warm to room temperature. After 20 minutes, the reaction mixture was quenched with methanol, absorbed onto silica gel and then purified by silica gel chromatography to yield a mixture of cis-trans isomers (formula IB, Scheme 2). The cis-trans isomer mix IB was dissolved in methanol and 10% palladium on carbon (50% by weight of IB) was added. The mixture was placed under 50 psi hydrogen and was heated at 50° C. for 10 to 24 hours. The mixture was filtered through Celite and was purified by silica gel chromatography. The compounds of the following examples were prepared according to this general procedure using the corresponding phosphonate.

### EXAMPLE 3

### 4-(4-tert-Butyl-phenyl)-2-(4-methyl-3,4,5,6-tetrahydro-2H-[1,2′]bipyrazinyl-3′-ylmethyl)-morpholin-3-one

4-(4-tert-Butyl-phenyl)-2-(4-methyl-3,4,5,6-tetrahydro-2H-[1,2′]bipyrazinyl-3′-ylmethyl)-morpholin-3-one: ¹³C NMR (100 MHz, CDCl₃) d 169.6, 158.0, 150.1, 146.8, 139.6, 136.9, 126.4, 125.2, 112.5, 76.3, 63.6, 55.3, 50.3, 49.7, 46.4, 36.3, 34.8, 31.6; MS (AP/CI) 424.2 (M+H)+.

### EXAMPLE 4

### 1-(4-tert-Butyl-phenyl)-3-(4-methyl-3,4,5,6-tetrahydro-2H-[1,2′]biprazinyl-3′-ylmethyl)-piperidin-2-one

1-(4-tert-Butyl-phenyl)-3-(4-methyl-3,4,5,6-tetrahydro-2H-[1,2′]bipyrazinyl-3′-ylmethyl)-piperidin-2-one: ¹³C NMR (100 MHz, CDCl₃) d 172.8, 158.0, 149.5, 148.7, 141.2, 139.1, 136.7, 126.2, 125.8, 55.4, 51.8, 49.7, 46.4, 40.5, 35.8, 34.7, 31.6, 27.0, 23.0; MS (AP/CI) 422.4 (M+H)+. Enantiomers were separable chromatographically (90/10 heptane-ethanol; Chiralcel OJ, 10 cm×25 cm; 250 mL/min; t1=ca. 9 min; t2=ca. 24 min).

### EXAMPLE 5

### 3-(4-Methyl-3,4,5,6-tetrahydro-2H-[1,2′]bipyrazinyl-3′-ylmethyl)-1-[4-(tetrahydro-pyran-4-yl)-phenyl]-piperidin-2-one

3-(4-Methyl-3,4,5,6-tetrahydro-2H-[1,2′]bipyrazinyl-3′-ylmethyl)-1-[4-(tetrahydro-pyran-4-yl)-phenyl]-piperidin-2-one: ¹³C NMR (100 MHz, CDCl₃) d 172.9, 157.9, 148.6, 144.3, 142.0, 139.2, 136.8, 127.6, 126.4, 68.6, 55.2, 51.8, 49.5, 46.3, 41.4, 40.5, 35.7, 34.1, 26.9, 23.0; MS(AP/CI) 450.4 (M+H)+. The enantiomers were separable chromatographically (85/15 acetonitrile-methanol; Chiralpak AS, 10 cm×50 cm; 250 mL/min; t1=ca. 19 min; t2=ca. 25 minutes).

### PREPARATION 2

### 3-(4-methyl-3,4,5,6-tetrahydro-2H-[1,2′]bipyrazinyl-3′-ylmethylene)-piperidin-2-one

A solution of (2-oxo-piperidin-3-yl)-phosphonic acid diethyl ester (11.2 mmol, 2.6 g) in tetrahydrofuran (100 mL) cooled to 0° C. was treated dropwise with sodium bis(trimethylsilyl)amide (22.3 mmole, 22.3 mL, 1M in tetrahydrofuran). The cooling bath was then removed. After 30 minutes the solution was cooled to 0° C. and 4-methyl-3,4,5,6-tetrahydro-2H-[1,2′]bipyrazinyl-3′-carbaldehyde (11.2 mmole, 2.3 g) in tetrahydrofuran (20 mL) was added dropwise. The solution was stirred for 3 hours as the cooling bath expired. About 10 mL methanol was added and the reaction mixture was adsorbed onto silica gel and purified by silica gel chromatography (90:1 chloroform-methanol w/1% ammonium hydroxide solution) to yield 1.83 g (57%) of 3-(4-methyl-3,4,5,6-tetrahydro-2H-[1,2′]bipyrazinyl-3′-ylmethylene)-piperidin-2-one; ¹³CNMR (100 MHz, CDCl₃) d 165.53, 155.90, 144.06, 139.99, 135.75, 133.67, 55.34, 55.13, 49.00, 46.45, 42.45, 31.55, 23.38; MS (AP/CI) 288.4 (M+H)+.

### PREPARATION 3

### 3-(4-methyl-3,4,5,6-tetrahydro-2H-[1,2′]bipyrazinyl-3′-ylmethyl)-piperidin-2-one

A mixture containing 3-(4-methyl-3,4,5,6-tetrahydro-2H-[1,2′]bipyrazinyl-3′-ylmethylene)-piperidine-2-one (6.27 mmol, 1.83 g) and 1 g of 10% palladium on carbon in 40 mL methanol was placed under 50 psi hydrogen and heated to 50° C. for 24 hours. After cooling to room temperature, the mixture was filtered through Celite™. The filtrate was concentrated in vacuo to yield 1.78 g of 3-(4-methyl-3,4,5,6-tetrahydro-2H-[1,2′]bipyrazinyl-3′-ylmethyl)-piperidin-2-one; ¹³C NMR (100 MHz, CDCl₃) d 174.89, 157.99, 148.48, 139.19, 135.93, 55.25, 49.60, 46.30, 42.71, 39.78, 35.26, 26.48, 22.12; MS (AP/CI) 290.4 (M+H)+.

### EXAMPLE 6

### 3-(4-methyl-3,4,5,6-tetrahydro-2H-[1,2′]bipyrazinyl-3′-ylmethyl)-1-[4-(4-methyl-tetrahydro-pyran-4-yl)-phenyl]piperidin-2-one

A mixture of 3-(4-methyl-3,4,5,6-tetrahydro-2H-[1,2′]bipyrazinyl-3′-ylmethyl)-piperidin-2-one (0.865 mmol, 250 mg), 4-(4-bromo-phenyl)-4-methyl-tetrahydropyran (1.73 mmol, 441 mg) copper (I) iodide (1.73 mmol, 328 mg), N,N′-ethylenediamine (1.73 mmol, 184 μL), and potassium carbonate (1.3 mmol, 180 mg) in toluene (4 mL) was heated at 120° C. for 24 hours. The mixture was cooled to room temperature, the solids were filtered off, and the resultant solution was concentrated in vacuo and was purified by silica gel chromatography (100:1 chloroform-methanol w/1% ammonium hydroxide) and then by HPLC (Waters Symmetry 30×50 mm; 40 mL/min; 210 nM; 8 minute gradient, 9:1 water-acetonitrile w/0.1% formic acid to 1:1 water-acetonitrile w/0.1% formic acid; approximate retention time=3.9 minutes). After removal of the water-acetonitrile solvent, the residue was dissolved in methylene chloride, washed with aqueous sodium hydroxide (1N), dried over magnesium sulfate, filtered and the solvent removed in vacuo to yield 125 mg (31%) of the title compound; ¹³C NMR (100 MHz, CDCl₃) d 172.9 158.0, 148.6, 174.2 141.5, 139.1, 136.7, 126.5, 126.2, 64.6, 55.3, 51.7, 49.6, 46.4, 40.5, 37.85, 37.81, 35.7, 29.3, 26.9, 23.0; MS (AP/CI) 464.5 (M+H)+.

1. A compound of the formula 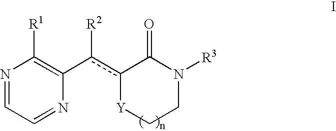
wherein R¹ is a group of the formula G¹ or G² depicted below, 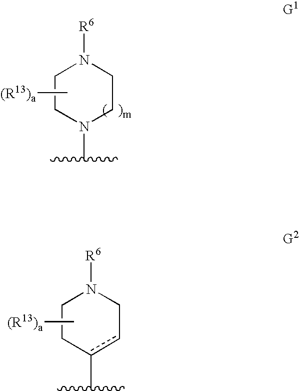
wherein R⁶ is hydrogen or —C(═O)—OR wherein R is C₁-C₈ straight chain or branched alkyl, C₃-C₈ cycloalkyl, or aryl; or
R⁶ is (C₁-C₆)alkyl or (C₁-C₄)alkyl-aryl wherein said aryl moiety is phenyl or naphthyl, optionally substituted with one or more substituents independently selected from (C₁-C₆)alkyl, (C₁-C₆)alkoxy, trifluoromethyl, cyano and SO_{g}(C₁-C₆)alkyl wherein g is zero, one or two;
each R¹³ is, independently, hydrogen, (C₁-C₄)alkyl, benzyl, or a (C₁-C₄)alkylene bridge from one of the ring carbons of the piperazine ring of G¹ to a ring carbon of the same ring or another ring or to a ring nitrogen of the piperazine ring having an available bonding site, or to a ring carbon of R⁶, when R⁶ has a ring structure having an available bonding site or a (C₁-C₄)alkylene bridge from one of the ring carbons of the piperidine ring of G² to a ring carbon of the same ring or another ring or to an amine substituent of the piperidine ring having an available bonding site, or to a ring carbon of R⁷ or R⁸, when either of R⁷ or R⁸ has a ring structure having an available bonding site;
a is zero to eight;
m is one, two or three;
Y is carbon, sulfur, nitrogen or oxygen;
R is hydrogen, (C₁-C₆)alkyl, or benzyl;
R³ is vinyl, C(═O)R, wherein R is straight chain or branched (C₁-C₈)alkyl, (C₃-C₈)cycloalkyl, trifluoromethyl, or aryl; or,
R³ is —(CH₂)_{g}B, wherein g is zero to three and B is hydrogen, phenyl, naphthyl or a 5 to 7-membered heteroaryl ring containing from one to four heteroatoms in the ring selected from oxygen, nitrogen and sulfur, with the proviso that said ring cannot contain two adjacent oxygen atoms or two adjacent sulfur atoms and wherein the foregoing phenyl, naphthyl and heteroaryl rings may optionally be substituted with one to three substituents independently selected from chloro, fluoro, bromo, iodo, aryl-O—, heteroaryl-O—, aryl(C═O), heteroaryl(C═O), (C₁-C₈)alkyl, (C₁-C₈)hydroxyalkyl-, (C₁-C₈)alkoxy, (C₁-C₈)alkoxy-(C₁-C₈)alkyl-, (C₃-C₈)cycloalkyl-, (C₃-C₈)hydroxycycloalkyl, (C₃-C₈)cycloalkyl-O—, and wherein one to three carbon atoms of each of the foregoing (C₃-C₈)cycloalkyl substituents may be replaced with a heteroatom independently selected from nitrogen, oxygen or sulfur to form a heterocycloalkyl substituent having 4 to 8 atoms, with the proviso that said heterocycloalkyl substituent cannot contain two adjacent oxygen atoms or two adjacent sulfur atoms, and wherein each (C₃-C₈)cycloalkyl or heterocycloalkyl substituent may be independently substituted with from zero to three substituents independently selected from (C₁-C₈)alkyl, (C₁-C₄)alkyl-aryl wherein said aryl moiety is phenyl or naphthyl, hydroxy, and (C₁-C₈)alkoxy;
wherein when B is phenyl, naphthyl or heteroaryl, B may be optionally substituted with zero to three substituents independently selected from phenyl, naphthyl or a 5 to 7-membered heteroaryl ring containing from one to four heteroatoms selected from oxygen, nitrogen and sulfur, with the proviso that said heteroaryl ring cannot contain two adjacent oxygen atoms or two adjacent sulfur atoms, and wherein each independently selected phenyl, naphthyl or heteroaryl substituent may itself be independently substituted with from zero, one, two or three (C₁-C₈)alkyl or halo substituents; or, B may be optionally substituted with from zero to three substituents independently selected from nitro, trifluoromethyl, trifluoromethoxy, cyano, hydroxy, —CH₂OH, —COOH or the lactone formed from hydroxy or —CH₂OH with an ortho —COOH, and —SOₜ(C₁-C₆)alkyl wherein t is zero to two, or —CONR¹⁴R¹⁵, wherein R¹⁴ and R¹⁵ are independently selected from (C₁-C₈)alkyl, benzyl, or R¹⁴ and R¹⁵ together with the nitrogen to which they are attached form a 5 to 7-membered heteroalkyl ring that may contain from zero to three heteroatoms selected from nitrogen, sulfur and oxygen in addition to the nitrogen of the —CONR¹⁴R¹⁵ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms, or —(CH₂)ᵥNCOR¹⁶R¹⁷ wherein v is zero to three and —COR¹⁶ and R¹⁷ taken together with the nitrogen to which they are attached form a 4 to 6-membered lactam ring;
n is zero, one or two;
wherein the broken line indicates an optional double bond;
or, a pharmaceutically acceptable salt thereof.

2. The compound according to claim 1 wherein R³ is (CH₂)_{g}B wherein g is zero and B is selected from phenyl and pyridyl.

3. The compound according to claim 2 wherein said phenyl or pyridyl has one to three substituents independently selected from: (C₁-C₈)alkyl, (C₃-C₈)cycloalkyl-, and (C₃-C₈)cycloalkyl-O—, wherein one, two or three carbon atoms of each of the foregoing (C₃-C₈)cycloalkyl substituents may be replaced with a heteroatom independently selected from nitrogen, oxygen and sulfur to form a heterocycloalkyl substituent having 4 to 8 atoms, with the proviso that said heterocycloalkyl substituent cannot contain two adjacent oxygen atoms or two adjacent sulfur atoms, and wherein each (C₁-C₈)alkyl, (C₃-C₈)cycloalkyl or heterocycloalkyl substituent may be independently substituted with one, two or three substituents independently selected from (C₁-C₈)alkyl, (C₁-C₄)alkyl-aryl, hydroxy, and (C₁-C₈)alkoxy, wherein said aryl moiety is phenyl or naphthyl.

4. The compound according to claim 2 wherein said phenyl or pyridyl has one, two or three substituents independently selected from: tetrahydropyranyl, morpholinyl, azetidinyl, pyrrolidinyl, piperidyl, piperazinyl, morpholinyl, thiomorpholinyl, hexahydroazepinyl, diazepinyl, oxazepinyl, thiazepinyl, oxadiazepinyl, thiadiazepinyl or triazepinyl, oxetanyl, and tetrahydrofuranyl, wherein each said substituent may be independently substituted with from one, two or three substituents independently selected from (C₁-C₈)alkyl.

5. The compound according to claim 2 wherein said phenyl or pyridyl is substituted with one, two or three substituents independently selected from pyridyl, pyrrolyl, pyrazolyl, imidazolyl, triazolyl, tetrazolyl, oxazolyl and oxadiazolyl.

6. The compound according to claim 1 wherein Y is carbon or oxygen and n is zero or one.

7. The compound according to claim 1 wherein R⁶ is selected from hydrogen, (C₁-C₆)alkyl, (C₁-C₄)alkyl-aryl, and —C(═O)—O(C₁-C₈)alkyl, wherein said aryl moiety is phenyl or naphthyl; R¹³ is (C₁-C₈)alkyl; a is zero to three; and, m is one.

8. The compound according to claim 1 wherein R⁶ is selected from hydrogen, methyl, ethyl and benzyl; R¹³ is methyl; a is zero, one or two; m is one; and, n is zero or one.

9. The compound according to claim 1 selected from the group consisting of
1-(4-tert-Butyl-phenyl)-3-(4-methyl-3,4,5,6-tetrahydro-2H-[1,2′]bipyrazinyl-3′-ylmethyl)-pyrrolidin-2-one;
3-(4-Methyl-3,4,5,6-tetrahydro-2H-[1,2′]bipyrazinyl-3′-ylmethyl)-1-[4-(tetrahydro-pyran-4-yl)-phenyl]-pyrrolidin-2-one;
4-(4-tert-Butyl-phenyl)-2-(4-methyl-3,4,5,6-tetrahydro-2H-[1,2′]bipyrazinyl-3′-ylmethyl)-morpholin-3-one;
1-(4-tert-Butyl-phenyl)-3-(4-methyl-3,4,5,6-tetrahydro-2H-[1,2′]bipyrazinyl-3′-ylmethyl)-piperidin-2-one;
3-(4-Methyl-3,4,5,6-tetrahydro-2H-[1,2′]bipyrazinyl-3′-ylmethyl)-1-[4-(tetrahydro-pyran-4-yl)-phenyl]-piperidin-2-one;
3-(4-Methyl-3,4,5,6-tetrahydro-2H-[1,2′]bipyrazinyl-3′-ylmethylene)-piperidin-2-one;
3-(4-Methyl-3,4,5,6-tetrahydro-2H-[1,2′]bipyrazinyl-3′-ylmethyl)-piperidin-2-one;
3-(4-Methyl-3,4,5,6-tetrahydro-2H-[1,2′]bipyrazinyl-3′-ylmethyl)-1-[4-(4-methyl-tetrahydro-pyran-4-yl)-phenyl]piperidin-2-one;
(+)-1-(4-tert-Butyl-phenyl)-3-(4-methyl-3,4,5,6-tetrahydro-2H-[1,2′]bipyrazinyl-3′-ylmethyl)-pyrrolidin-2-one;
(+)-3-(4-Methyl-3,4,5,6-tetrahydro-2H-[1,2′]bipyrazinyl-3′-ylmethyl)-1-[4-(tetrahydro-pyran-4-yl)-phenyl]-pyrrolidin-2-one;
(+)-4-(4-tert-Butyl-phenyl)-2-(4-methyl-3,4,5,6-tetrahydro-2H-[1,2′]bipyrazinyl-3′-ylmethyl)-morpholin-3-one;
(+)-1-(4-tert-Butyl-phenyl)-3-(4-methyl-3,4,5,6-tetrahydro-2H-[1,2′]bipyrazinyl-3′-ylmethyl)-piperidin-2-one;
(+)-3-(4-Methyl-3,4,5,6-tetrahydro-2H-[1,2′]bipyrazinyl-3′-ylmethyl)-1-[4-(tetrahydro-pyran-4-yl)-phenyl]-piperidin-2-one;
(+)-3-(4-methyl-3,4,5,6-tetrahydro-2H-[1,2′]bipyrazinyl-3′-ylmethyl)-piperidin-2-one;
(+)-3-(4-methyl-3,4,5,6-tetrahydro-2H-[1,2′]bipyrazinyl-3′-ylmethyl)-1-[4-(4-methyl-tetrahydro-pyran-4-yl)-phenyl]piperidin-2-one;
(−)-1-(4-tert-butyl-phenyl)-3-(4-methyl-3,4,5,6-tetrahydro-2H-[1,2′]bipyrazinyl-3′-ylmethyl)-pyrrolidin-2-one;
(−)-3-(4-Methyl-3,4,5,6-tetrahydro-2H-[1,2′]bipyrazinyl-3′-ylmethyl)-1-[4-(tetrahydro-pyran-4-yl)-phenyl]-pyrrolidin-2-one;
(−)-4-(4-tert-Butyl-phenyl)-2-(4-methyl-3,4,5,6-tetrahydro-2H-[1,2′]bipyrazinyl-3′-ylmethyl)-morpholin-3-one;
(−)-1-(4-tert-Butyl-phenyl)-3-(4-methyl-3,4,5,6-tetrahydro-2H-[1,2′]bipyrazinyl-3′-ylmethyl)-piperidin-2-one;
(−)-3-(4-Methyl-3,4,5,6-tetrahydro-2H-[1,2′]bipyrazinyl-3′-ylmethyl)-1-[4-(tetrahydro-pyran-4-yl)-phenyl]-piperidin-2-one;
(−)-3-(4-Methyl-3,4,5,6-tetrahydro-2H-[1,2′]bipyrazinyl-3′-ylmethyl)-piperidin-2-one;
(−)-3-(4-Methyl-3,4,5,6-tetrahydro-2H-[1,2′]bipyrazinyl-3′-ylmethyl)-1-[4-(4-methyl-tetrahydro-pyran-4-yl)-phenyl]piperidin-2-one; and,
pharmaceutically acceptable salts thereof.

10. A pharmaceutical composition comprising a compound according to claim 1, or a pharmaceutically acceptable salt thereof, and a pharmaceutically acceptable carrier.

11. A method of treating a disorder or condition in a mammal selected from depression, anxiety, depression with concomitant anxiety, post traumatic stress disorder, panic phobias, obsessive compulsive disorder (OCD), borderline personality disorder, sleep disorder, psychosis, seizures, dyskinesis, symptoms of Huntington's or Parkinson's diseases, spasticity, suppression of seizures resulting from epilepsy, cerebral ischemia, anorexia, faintness attacks, hypokinesia, cranial traumas, chemical dependencies, premature ejaculation, premenstrual syndrome (PMS) associated mood and appetite disorder, inflammatory bowel disease, modification of feeding behavior, blocking carbohydrate cravings, late luteal phase dysphoric disorder, tobacco withdrawal-associated symptoms, panic disorder, bipolar disorder, sleep disorders, jet lag, cognitive dysfunction, hypertension, bulimia, anorexia, obesity, cardiac arrhythmias, chemical dependencies and addictions selected from dependencies on, or addictions to nicotine or tobacco products, alcohol, benzodiazepines, barbiturates, opioids or cocaine; headache, stroke, traumatic brain injury (TBI), psychosis, Huntington's Chorea, tardive dyskinesia, hyperkinesia, dyslexia, schizophrenia, multi-infarct dementia, epilepsy, senile dementia of the Alzheimer's type (AD), Parkinson's disease (PD), attention deficit hyperactivity disorder (ADHD) and Tourette's Syndrome, comprising administering to a mammal in need of such treatment an amount of the compound according to claim 1, or a pharmaceutically acceptable salt thereof, that is effective in treating such disorder or condition.

12. A method of treating a disorder or condition in a mammal selected from depression, anxiety, depression with concomitant anxiety, post traumatic stress disorder, panic phobias, obsessive compulsive disorder (OCD), borderline personality disorder, sleep disorder, psychosis, seizures, dyskinesis, symptoms of Huntington's or Parkinson's diseases, spasticity, suppression of seizures resulting from epilepsy, cerebral ischemia, anorexia, faintness attacks, hypokinesia, cranial traumas, chemical dependencies, premature ejaculation, premenstrual syndrome (PMS) associated mood and appetite disorder, inflammatory bowel disease, modification of feeding behavior, blocking carbohydrate cravings, late luteal phase dysphoric disorder, tobacco withdrawal-associated symptoms, panic disorder, bipolar disorder, sleep disorders, jet lag, cognitive dysfunction, hypertension, bulimia, anorexia, obesity, cardiac arrhythmias, chemical dependencies and addictions selected from dependencies on, or addictions to nicotine or tobacco products, alcohol, benzodiazepines, barbiturates, opioids or cocaine; headache, stroke, traumatic brain injury (TBI), psychosis, Huntington's Chorea, tardive dyskinesia, hyperkinesia, dyslexia, schizophrenia, multi-infarct dementia, epilepsy, senile dementia of the Alzheimer's type (AD), Parkinson's disease (PD), attention deficit hyperactivity disorder (ADHD) and Tourette's Syndrome, comprising administering to a mammal in need of such treatment an amount of the compound according to claim 1 that is an effective antagonist, inverse agonist or partial agonist of 5-HT_{1A} or 5-HT_{1B} receptors or a combination of 5-HT_{1A} and 5-HT_{1B} receptors.
